(19) United States
(12) Patent Application Publication
(10) Pub. No.: US 2023/0130240 A1
(43) Pub. Date: Apr. 27, 2023

(54) Trocar Device and System and Method for Monitoring Navigation of the Trocar Device

(71) Applicant: OttoSurgical Instruments LLC, Los Angeles, CA (US)

(72) Inventor: Gary D. Ott, Los Angeles, CA (US)

(21) Appl. No.: 17/507,570

(22) Filed: Oct. 21, 2021

(51) Int. Cl. A61B 17/34; A61B 34/20; A61B 34/30

(52) U.S. Cl. CPC A61B 17/3468; A61B 34/20; A61B 34/30; A61B 2562/0271; A61B 2562/0247; A61B 2017/00805

(57) ABSTRACT

An improved trocar device or instrument configured for single or multiple uses is disclosed. The improved trocar device has a novel flexible surgical assembly including an introducer component or a handle with scissor-like members with a channel formed within to accommodate a shaft component that slides within the channel. The introducer component may be squeezed or compressed to cause a locking mechanism to engage the shaft component to secure it and maintain it steady near the active area of the surgical procedure. The improved trocar device or instrument is coupled to a monitoring system for automatically monitoring the penetration route and behavior of the trocar device held by a robotic arm or a surgeon to assist either one with navigating the trocar shaft into a patient's body.

### BACKGROUND

### 1. Field of the Invention

The present invention relates generally to the field of surgical instruments. In particular, the present invention relates to an improved trocar device used for introducing a mid-urethral sling for treating female urinary incontinence, i.e., incapacity of controlling the discharge of urine. The improved trocar device has two components, a flexible introducer or handle and a shaft component, with the flexible introducer configured to guide the shaft component into a patient's body.to position the mid-urethral sling in a desired location. The improved trocar device is configured for either single use or multiple uses. The present invention also relates to a system and method for dynamically monitoring navigation of the improved trocar device as it held either by a surgeon or a robot art and introduced into a patient's body.

### 2. Description of the Related Art

Incontinence among women is common, especially as women age. The majority of women suffer from stress urinary incontinence (“SUI”). Women with SUI involuntarily discharge urine during usual daily activities and movements, for example, laughing, coughing, sneezing and during regular exercise. Over the decades, surgeons have introduced dozens of procedures to ease stress incontinence.

The cause of stress urinary incontinence (“SUI”) can be a functional defect or weakening of the tissue or ligaments that connect the vaginal wall with the pelvic muscles and the pubic bone. Normally, the urethra, when supported by strong muscles of the pelvic floor and healthy connective tissue, maintains a watertight seal that prevents the involuntary discharge of urine. However, when a woman suffers from the most common type of stress urinary incontinence (“SUI”), weakened muscle and pelvic tissues cannot adequately support the urethra in its correct position. Consequently, during normal movements, when pressure is exerted on the bladder from the diaphragm, the urethra cannot retain its seal and urine escapes. Since the stress urinary incontinence is annoying and unpredictable, many women who suffer from it avoid an active lifestyle and social situations.

Common causes include repetitive pelvic muscle strain, delivery, loss of pelvic muscle tone, and loss of estrogen. This defect causes the improper functioning of the urethra. Unlike other types of incontinence, stress urinary incontinence is not a bladder problem. To elaborate, the bladder lies in front of the vagina. A layer of smooth muscle in the bladder wall, called the “detrusor,” allows the bladder to expand and store urine. Extending from the bladder to the outside of the body is the urethra, a slender, flexible tube that is also surrounded by smooth muscle. At the junction where the urethra meets the bladder (the “bladder neck”), two sets of sphincter muscles (the “internal urethral sphincter” and the “external sphincter”) help hold urine in. A network of muscles and ligaments, known collectively as the “pelvic floor,” supports the entire urinary system, the reproductive organs, and the intestines. The pelvic floor extends from the pubic bone to the tailbone, with openings for the urethra, vagina, and anus. To avoid urinating, a woman tightens the muscles of her pelvic floor and the external sphincter.

Many activities, including sneezing and lifting a heavy object, increase pressure on the bladder and throughout the abdomen. If this results in urine leakage, a woman experiences stress incontinence. The most common source of the problem is urethral hypermobility—the bladder and urethra shifting downward as abdominal pressure rises. If pelvic muscles have been weakened, the urethra will open and urine can leak out. The risk is greater not only for women who are older, but obese, or have given birth vaginally many times. A less common type of anatomical problem that can lead to incontinence is intrinsic sphincter deficiency, in which sphincter muscles don't close completely or can't prevent the sphincter from opening under physical pressure. Intrinsic sphincter deficiency usually results from injury during surgery (sometimes undertaken to address urethral hypermobility) or from general tissue atrophy in the pelvic region.

Until fairly recently, the accepted standard for incontinence surgery was the Burch procedure, or Burch colposuspension. This procedure lifts up the tissue near the bladder neck to increase its support during physical exertion. After making an incision in the abdomen along the bikini line, the surgeon places strong stitches into the tissue of the vaginal wall at the level of the bladder neck and upper urethra and secures them to a ligament near the top of the pubic bone. However, the Burch procedure is performed under general anesthesia and usually requires a two-day hospital stay and six weeks of recovery before a patient can return to full activity.

Most procedures for treating stress incontinence are designed to support the urethra and bladder neck. In the Burch procedure, support is provided by strong stitches (“sutures”) extending on both sides of the bladder neck from the vaginal wall to a ligament near the pubic bone. Sling procedures use a patient's own tissue (fascial sling) or a synthetic tape or mesh (for example, tension-free vaginal tape) to provide support.

Another well-established procedure for stress incontinence caused by urethral hypermobility is the fascial sling. In this case, the surgeon makes an incision in the lower abdomen, takes a thin strip of ligament from the abdominal wall, and uses it to create a U-shaped sling to support the upper urethra and hold it in place. The sling is inserted through an incision in the vagina and the ends are fastened to the abdominal wall at the top of the pubic bone. Again, this procedure is performed under general or regional anesthesia and typically requires an overnight hospital stay. Recovery normally takes at least a couple of weeks.

Although the Burch procedure and the fascial sling both have long track records and reported success rates of 60% to 90%, the sling procedure is generally more effective. Yet, women undergoing the sling procedure were more likely to develop urinary tract infections and more likely to be discharged from the hospital with a catheter. They were also more likely to develop urge incontinence and long-term difficulty urinating. In some instances, women with slings require further surgery to alleviate urinary retention.

In recent years, a third alternative has been introduced, namely, inserting a sling made of synthetic material, such as nylon mesh. This procedure typically does not require general anesthesia or hospitalization. The sling stays in place without sutures because the body's own tissue grows through the mesh and supports it.

There are several devices and methods that exist for inserting the sling made of synthetic material. Some involve a surgical instrument comprising a shank having a handle at one end and connecting means at the other end for receiving, one at a time, two curved needle-like elements. Each is connected at one end to opposing ends of a mesh that is implanted in a woman's body. In practice, the synthetic mesh is introduced into the body through the vagina first at one end and then at the other end, on one side and the other, respectively, of the urethra, to form a support around the urethra located between the urethra and the vaginal wall. The mesh is positioned to extend over the pubis and through the abdominal wall and tenses. The ends of the mesh are cut in the abdominal wall and the mesh is thereby implanted in the body. This procedure known as “Transvaginal” is illustrated by the TYT product marketed by the Gynecare franchise of Ethicon Inc., a Johnson & Johnson Company, of Somerville, N.J., USA. In this procedure, two 5 mm needles traverse a PROLENE mesh trans-vaginally and through the abdomen, to create a tension-free support beneath the middle urethra.

In the tension-free vaginal tape procedure (“TVT”), a mesh sling is inserted through an incision in the vagina with a surgical instrument about as wide as a pencil. The mesh is positioned in a U shape under and around the urethra and its ends are brought up between the bladder and pubic bone and out through tiny abdominal incisions above the pubic bone. The sling supports the bladder neck and urethra, helping the urethra hold in urine during a cough or sneeze. In an alternative procedure, called SPARC, for “suprapubic arch,” the sling is inserted from the top down, entering through small incisions in the abdomen above the pubic bone and exiting through the vagina. This approach may pose less risk to the bladder and nearby blood vessels.

A newer sling, the transobturator tape (“TOT”) procedure, avoids the space between the pubic bone and the bladder, reducing the risk of injury to the area during surgery. Instead, the supporting mesh is inserted through the vagina and the ends are brought out through tiny incisions between the labia and the creases of the thighs. TOT requires no abdominal incisions and is shaped more like a smile than a U, which is found to be gentler on the urethra than TVT. The TOT procedure prevents bladder perforation. Other complications may include urinary retention, leg pain or difficulty in walking, and erosion of the mesh through the vaginal lining.

Another different method has been used to place the sub-urethral slings in which a needle first traverses the abdominal wall along the same path, as described above and, eventually, exits through the vaginal incision. The tape is then attached in some way to the needle and pulled through the body from the vaginal incision outward through the abdominal incision. This procedure is referred to as “TransAbdominal” procedure. The chosen method, whether vaginal or abdominal, often depends on the preferences of each surgeon.

Yet another method for implanting a sub-urethral sling involves positioning the implanted sling to extend from below the urethra and outward through the obturator hole on each side. This procedure known as the “transobturator” procedure typically involves inserting a properly configured needle from a vaginal incision and, subsequently, outwards through the obturator orifice or vice versa. This technique can be performed with a surgical instrument that includes a surgical pin or introducer elements and tubes applied over the ends of the surgical pins coupled with the tape that will be implanted below the urethra.

Many incontinence surgeries must be performed at least partially “blind,” hence a surgeon's complete familiarity with the anatomy is essential. During the surgical procedure, the surgeon opens up the midparaurethral space for passing through the “vaginal trocar” in the retropubic space. Several issues typically arise. The patient's position on the operating table should result in placement of the symphysis pubis in a near-vertical plane; this usually means keeping the patient's torso and head in a level horizontal position or slightly up in a reverse Trendelenburg position. Small 5-mm stab incisions are made through the abdominal skin overlying the top of the symphysis approximately 2.5 cm from the midline on either side. This maneuver allows easier manipulation of the ligature carrier for the antegrade approach or “vaginal trocars” for the retrograde approach by avoiding any resistance at the skin level. It also provides a visual location for avoiding the perforation of the skin and rectus fascia too laterally because that may lead to the reported complication of ilioinguinal nerve or inferior epigastric vascular damage. For the tension-free vaginal tape (TVT) procedure, the vaginal trocars are typically placed in the anterior vaginal wall pocket. After it is “seated” in the pocket (˜2-3 cm), the trocar tip is turned toward the ipsilateral shoulder and guided under the pubis with the nondominant hand. By cradling the trocar with the nondominant hand and holding the handle with the dominant hand, the trocar is gently advanced in an upward fashion behind the posterior aspect of the symphysis and out through the abdominal stab wounds. Attention to staying on the posterior or back side of the symphysis is required to prevent entering the rectus muscle and fascia too far cephalad from its insertion on the symphysis. Removal of the weighted speculum from the vagina and cradling the curve of the trocar in the nondominant hand ensures some control of the device and helps to guide the device in a straight upward direction as pressure is applied with both hands. When using the percutaneous ligature carrier, the free sling is fashioned from a large sheet of polypropylene mesh (e.g., PML, Ethicon, Somerville, N.J.) to approximately the same size as supplied in the TVT kit and the ligature carrier is passed down in an antegrade fashion to the urethropelvic complex at the location described above for locating the insertion of the vaginal trocars for the TVT procedure. To guide the ligature carrier into the vagina, the periurethral pocket must be developed further toward the arcus tendineus to allow entrance of a finger to guide the carrier out and into the vagina. The suspending sutures of the tape are then threaded into the carrier and retrieved at the abdominal skin site. Cystoscopy is performed to inspect for foreign body material within the urinary tract, and it is best to do this inspection when the ligature carrier for PVT or vaginal trocar for TVT is still within the retropubic space. Inspection must take place with a full bladder and use of the 70° lens. A clue that a perforation of the bladder has taken place is discovering cystoscopic fluid extravasation around the trocar or the sling material at the level of the abdominal skin. If the ligature carrier or vaginal trocar device has been placed through the bladder, the surgeon has to extract the device to try again.

At the end of most procedures, the surgeon must use a lighted medical scope to inspect the inside of the bladder for injury. The risk of injuring the bladder require surgeons to leave a urethral catheter for several extra days to ensure bladder healing of the injury to avoid any chance that postoperative urinary retention would lead to urinary extravasation. Bladder perforations are more common, but trocar injury of the urethra can also occur. In the event urethral perforation does occur, the procedure is terminated and the patient must return at a later date to repeat the surgery.

Thus, although the existing trocars currently used for mid-urethral sling procedures are more advanced than before, they still present challenges to surgeons. Even more, those with less experience have greater difficulty, as existing trocar devices are rigid and inflexible. Surgeons must grasp the trocar device at a proximal end distant form the distal end that enters the tissue while trying to maneuver it within a patient's body to position. The design of the current trocar devices has limitations. The surgeon typically must use both hands to ease the trocar into the body, carefully and slowly maneuvering it to avoid critical body structures, to avoid injuring the patient.

Moreover, current trocar devices are reusable, which adds the high cost of sterilization equipment, preventive maintenance, and replacement parts for the surgical devices and the sterilization equipment including sterilizer door gaskets, trays, and filters. As surgical instruments become more specialized and complicated, sterilization presents challenges. In addition, reusable surgical devices impact patient safety by introducing a risk of infection.

In addition, many strides to provide automated and mechanical assistance by robots have been made. For example, medical technology endoscopic robotic systems with robotic arms exist to assist the physician or surgeon during a surgical procedure. Robots serve as a guide, carrier, and instrument holder. The motorized drives of the robotic system allow movements and positionings with a high repetitive accuracy while significantly relieving the burden on the surgeon. Robotic systems guide and move robotic arms by an operator's touch. Typically, robots have previously only been used successfully where all parameters of the surroundings are fixed, predictable, or may be measured using the senses. During a manually conducted procedure or one in which a robot assists, during entry into a body cavity of a patient, for example, the abdominal area, the surgeon must exercise extreme caution. An incorrect or inordinate application of force may result in possible complications. Without knowledge of the effects of the trocar's movement as it moves into the patient's body, the surgeon must manually and completely move or control the instrument.

Therefore, to address growing demand for better surgical devices and monitoring systems, as healthcare expands in the world, it would be desirable to provide an improved surgical trocar device (for single use or reusable) with a flexible assembly and a monitoring system that can assist either the surgeon or a robot arm or system navigate the trocar device to effectively perform a surgical procedure such as position the mid-urethral sling. A flexible trocar device and monitoring system would enable a surgeon or a robot to easily guide and orient it during the surgical procedure with minimal risk of injury. It would also be desirable to manufacture a disposable or single use trocar device to eliminate patient-to-patient infections or a reusable trocar device that is more effective. There is a continuing need in the medical field for improved medical devices, surgical tools and instruments, and monitoring systems to reduce the risk of invasive procedures.

### SUMMARY

The present invention overcomes the deficiencies and limitations of prior trocar devices and methods for use, at least in part by, providing an improved trocar device with a flexible configuration, for single use in some embodiments, or multiple uses in other embodiments. All embodiments described here provide a surgeon or a robot greater flexibility to control and maneuver the trocar device while performing mid-urethral sling procedures. The single-use trocar device provides greater protection for patients, is available for immediate use, and is cost-efficient by eliminating the high cost of sterilization equipment, preventive maintenance, and replacement parts for the surgical devices and the sterilization equipment.

In accordance with some embodiments, the improved, single-use trocar device has at least two components including an introducer or handle and a needle-like shaft or shank (hereinafter referred to as a “shaft”). In some embodiments of the present invention, the introducer is made from plastic or like material and the shaft is made from stainless steel or like material, both designated for single use in accordance with some embodiments. The introducer or handle is configured to slide or move along the needle-like shaft or shank. Its flexible movement along the shaft allows the surgeon to move the introducer as desired and to position it closer to the active end of the trocar device as it is positioned to enter the patient's body. This improved flexible configuration permits the surgeon to control the direction and magnitude of force with greater ease while navigating the shaft of the improved trocar device. As described above, navigating the improved trocar properly without risk of injury to the patient is critical to appropriate positioning of the mesh prosthetic or “sling” while performing mid-urethral procedures on women.

The mesh arm is pulled by the trocar component hence it is critical that the movement of the trocar must be precisely navigated by the surgeon or a robot arm to avoid urologic, vascular, or visceral injuries that can occur while inserting the trocars. Increasing the accuracy and precision of trocar passage is of paramount importance. The improved trocar device advantageously provides greater control to ensure accuracy and precision for the surgeon. The surgeon's hand positioned closer to the active area allows the surgeon to more precisely control placement and positioning of the sling.

In accordance with one embodiment, the assembly of the improved single-use trocar device includes a scissor-like introducer and a needle-like shaft configured as a triangular part. In some embodiments, the assembly of the improved single-use trocar device is provided and salable as a kit, preferably with an introducer and two trocars that may be placed according to the OTT approach.

In accordance with some embodiments, the improved trocar device is made from materials that are suitable for multiple uses or to be reusable. For example, components may be made from stainless steel, anodized aluminum, and medical grade epoxy. In some embodiments, the introducer or handle may be made from a bio-compatible plastic material taken from a group including acetal, polyamide, polyethylene, polyvinylchloride; said plastic material being sterilizable at high temperature

In accordance with some embodiments, the improved trocar device or instrument is coupled to an instrument monitoring system for automatically and dynamically monitoring the penetration route of the improved trocar device as it is held and guided either by a surgeon or by a robotic arm. The method and system automatically monitor the penetration route and navigation behavior of the improved trocar device. In some embodiments, the improved trocar device includes a small pressure sensor at its tip, for example, embedded to provide continuous measurements as data to help the surgeon or robotic arm and/or an instrument guide the trocar into a body cavity of a patient during a surgical procedure. At least one measured value is recorded, by which a change in pressure or a force effect within the surface of the body of the patient may be determined, and automatic evaluation of the measured values with regard to a reference measured value is performed. Comparison of the change in the measured value or the change in the force effect with a threshold value is used to provide continuous indications to the surgeon or robotic arm, especially in the event of the threshold value being exceeded as generated as an output. In some embodiments, other sensors may be used such as to determine temperature or pH level.

### BRIEF DESCRIPTION OF THE DRAWINGS

The present invention of an improved trocar device configured in some embodiments for single use and in other embodiments for multiple uses is illustrated by way of example, and not by way of limitation, in the figures of the accompanying drawings in which like reference numerals are used to refer to the same or similar elements.

FIG. 1 illustrates a block diagram of an improved trocar device or instrument in accordance with a first embodiment of the present invention illustrating the introducer or handle component in solid lines and the shaft component in broken lines to illustrate the two different parts. The illustration in broken lines is for illustration purposes only.

FIG. 2 illustrates a bottom-rear-right perspective view of the first embodiment of the improved single-use trocar device or instrument in accordance with the present invention.

FIG. 3 illustrates a top view of the improved single-use trocar device or instrument of the first embodiment in accordance with the present invention. The shaft component is shown in broken lines to illustrate that the two components are separate.

FIG. 4 illustrates is a bottom view of the improved single-use trocar device in accordance with first embodiment of the present invention.

FIG. 5 illustrates a left-side elevational view of the improved single-use trocar device or instrument in accordance with one embodiment of the present invention. The positioning of the shaft component is illustrated in broken lines to illustrate that the two components are separate.

FIG. 6 illustrates a right-side elevational view of the first embodiment of the improved single-use trocar device in accordance with the present invention.

FIG. 7 illustrates a front-plan view of the first embodiment of the improved single-use trocar device or instrument in accordance with the present invention. The shaft component is illustrated in broken lines to demonstrate that the two components are separate.

FIG. 8 is a back-plan view of the first embodiment of the improved single-use trocar device in accordance with the present invention.

FIG. 9 is a top-front-left perspective view of an improved single-use trocar device or instrument with a shaft component shown in broken lines in accordance with a second embodiment of the present invention. This embodiment illustrates the channel opening within the introducer along the width of the middle portion of the introducer or handle.

FIG. 10 is a bottom-rear-right perspective view of the improved single-use trocar device or instrument.

FIG. 11 is a top view of the second embodiment of the improved single-use trocar or instrument with a shaft component in accordance with the second embodiment of the present invention. The shaft component is illustrated in broken lines.in accordance with the present invention.

FIG. 12 is a bottom view of the second embodiment of the improved single-use trocar in accordance with the present invention.

FIG. 13 is a left-side elevational view of the improved single-use trocar device or instrument in accordance with the present invention illustrating the shaft component in broken lines.

FIG. 14 is a right-side elevational view of the second embodiment of the improved single-use trocar device in accordance with the present invention.

FIG. 15 is a front-plan view of the second embodiment of the improved single-use trocar device or instrument with the shaft component (shown in broken lines) in accordance with the present invention.

FIG. 16 is a back-plan view of the second embodiment of the improved single-use trocar device in accordance with the present invention.

FIG. 17 is a perspective and partial cross-sectional view of the introducer and trocar shaft (in broken lines) components in accordance with the present invention.

FIG. 18 is a cross-sectional view of the introducer and trocar components of the improved single-use trocar device assembly in accordance with the present invention.

FIG. 19 is an illustration of one embodiment of a transvaginal trocar component in accordance with the present invention configured for bottom-up entry.

FIG. 20 is an illustration of another embodiment of a transabdominal trocar in accordance with the present invention configured for top-down entry.

FIG. 21 is an illustration of yet another embodiment of an obturator Foramen in accordance with the present invention.

FIG. 22 illustrates a high-level system block diagram of a graphical representation of the improved trocar device (according to any of the embodiments illustrated in FIGS. 1-21) couples to the instrument monitoring system in accordance with the present invention.

FIG. 23 is a flow chart illustrating the processes performed by the instrument measuring system coupled to the instrument monitoring system in accordance with the present invention.

It is to be understood that the attached drawings are for purposes of illustrating the concepts of the invention and may not be to scale, and are not intended to be limiting in terms of the range of possible shapes and/or proportions.

### DETAILED DESCRIPTION

Before explaining the present invention in detail, it should be recognized that the invention is not limited to this medical or surgical procedure, or use of all the details of the construction and configuration of the parts illustrated in the appended figures and the description. Illustrative embodiments of the invention may be implemented or incorporated into other modalities, variations and modifications, and may be practiced or carried out in various other ways. Although the improved trocar device configured for single use and multiple uses, is described for performance of mid-urethral sling procedures, this improved trocar device (or other surgical instruments or devices configured in a similar way) may be used in other vaginal procedures to address anterior vaginal wall supportive defects, posterior vaginal wall supportive defects, and apical vaginal wall supportive defects, each of which may be repaired by similar types of meshes; therefore, requiring a surgical tool for positioning such meshes. Other repair surgical procedures such as for hernia defects may also benefit from this type of a surgical assembly.

The single-use embodiment is otherwise referred to as a disposable device and it should be recognized by those skilled in the art that such device is intended for use on one patient during a single procedure; it is not intended to be reprocessed, by cleaning, disinfecting, or sterilizing and used on another patient. The multiple-use embodiment is also referred to as a reusable device and such embodiments must be sterilized according to a validated process used to render it free from viable microorganisms. The multiple-use embodiments require a level of reprocessing required for critical medical devices.

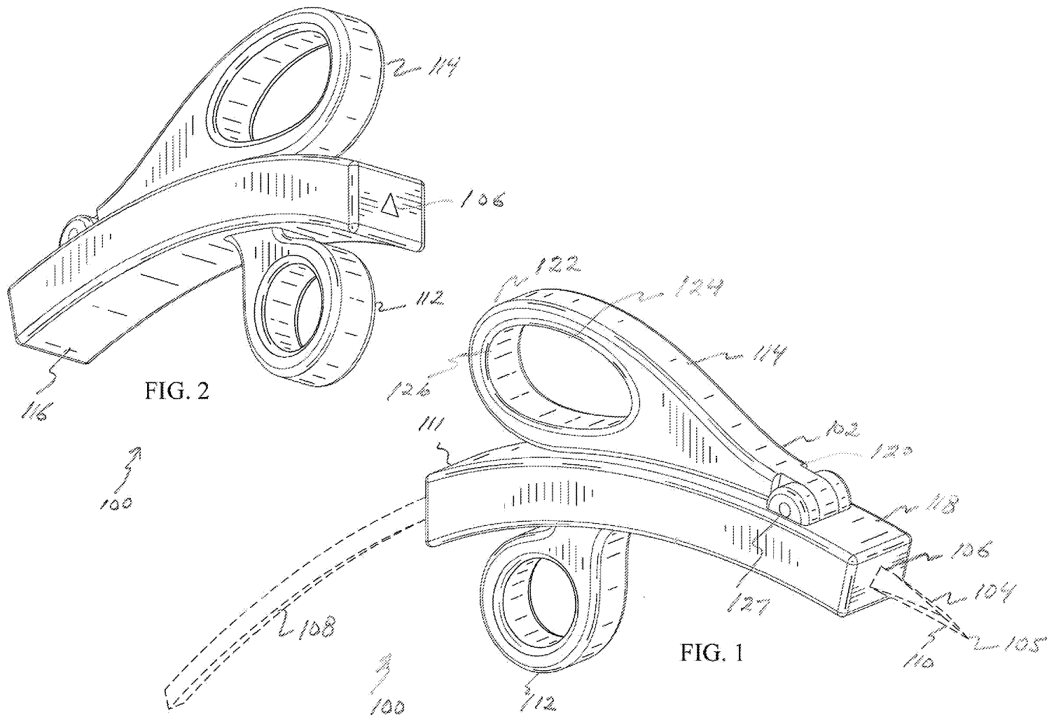

FIGS. 1-2 illustrate one embodiment of the improved trocar device designated by reference numeral 100 in accordance with the present invention. Referring to FIG. 1, the assembly of the improved trocar device 100, which may be configured for single use or multiple uses, comprises a surgical (trocar) introducer component 102, also referred to as a handle, designed to accommodate a trocar component 104, also referred to as a shaft, within a channel 106 created within the trocar introducer or handle 102, which allows the trocar component or shaft 104 to slide easily within the channel 106, also referred to as a groove. The improved trocar device 100 (for single use or reusable) in accordance with the present invention serves as an integrated delivery system with the trocar shaft 104 designed to maintain control during passage while reducing penetration force within a patient's body. The trocar device 100 features a smooth surface from handle or introducer 102 to closed tip 105, to reduce tissue drag during needle passage. The trocar curvature and tip radius are designed so that the trocar maintains contact with the posterior aspect of the pubic bone.

The surgical introducer component 102 serves as the hand grip portion for the surgeon or a robot arm and is preferably formed from a plastic material, although other materials may be suitable for use. The channel 106 is essentially a triangular orifice created and shaped to accommodate the trocar component or shaft 104, which is also triangular in shape. The channel 106 extends from an opening (to the triangular orifice) at the proximal end 108 to the closed distal end 110. The channel 106 within the trocar introducer component or handle 102 is shaped to conform to the triangular needle-like shaft 104 configured to enable the introducer component 102 slide up and down, essentially to permit guided and flexible movement along the length of the trocar shaft 104, from the proximal end 108 to the distal end 110. At the distal end 110, the trocar shaft 104 is configured to be introduced into the body through the vagina for implanting a mesh (not shown) in a woman's body. Referring now to FIGS. 1 and 2, the trocar introducer or handle 102 has a rectangular middle portion 111 with two scissor members 112 and 114, each attached to opposing walls 116 and 118, respectively, of the rectangular portion 111 as illustrated in the figures. The scissor members 112 and 114 are shaped like those in conventional surgical scissors with each scissor member having a distal portion 120 and a proximal handle portion 122. In the illustrated configuration, one of the two scissor members is larger as designated by reference numeral 114 and the other is smaller as designated by reference numeral 112. A finger opening 124 extends from a proximal end 126 of the proximal handle portion 122 in the direction of the tip of the trocar shaft 110. The scissor members 112 and 114 are pivotally connected via a pivot axis 127 (e.g., a pin) through the middle portion. As with conventional scissors, the scissor members 112 and 114 may be brought toward each other and squeezed together to prevent the trocar introducer or handle 102 from sliding. If the hold on the scissor members 112 and 114 is relaxed by a surgeon or robot arm, the trocar introducer 102 may be easily maneuvered to slide along the trocar shaft 104.

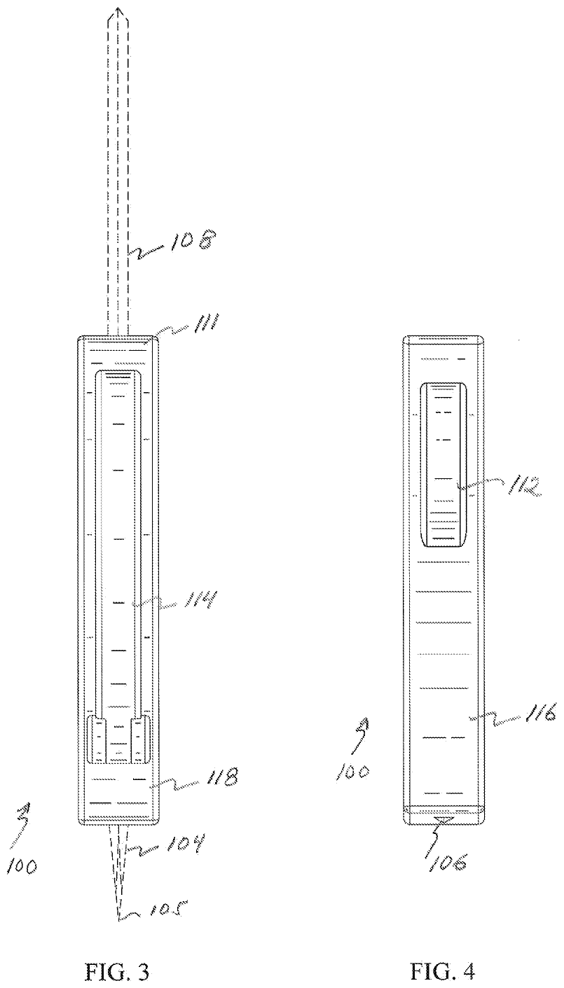

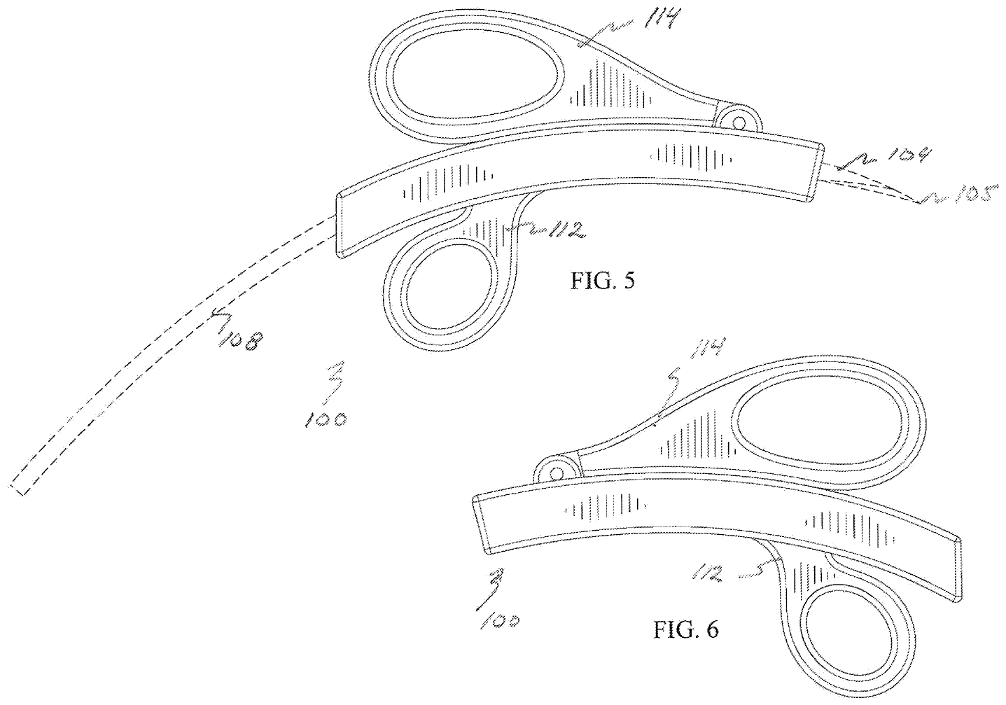

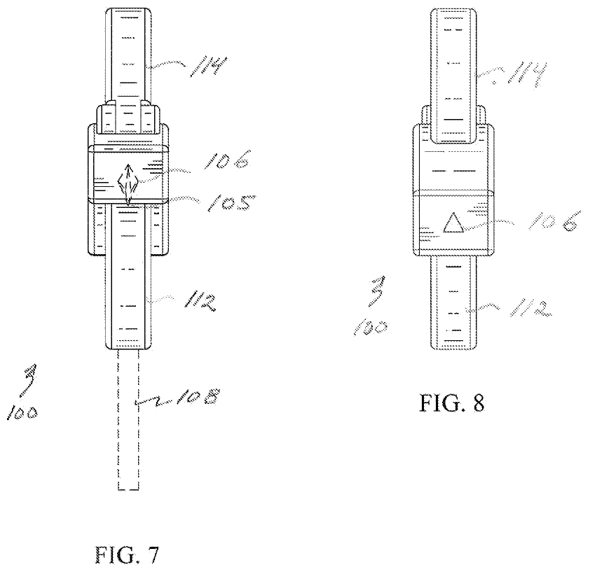

Referring now to FIGS. 3 and 4, the planar views of the trocar introducer or handler 102 and the trocar shaft 104 illustrate the dimensions of the scissor members 112 and 114 and orientation of the trocar shaft 104 within the trocar introducer 102, configured to enter through the triangular orifice 106. In the illustrated embodiment, the trocar shaft 104 is a needle element, which has an outer diameter of about 3.0 mm and the channel of the sheath element has a diameter of about 3.2 mm. The sheath element may also have an external diameter of approximately 4.2 mm. FIGS. 5 and 6 illustrate another view and FIGS. 7 and 8 yet another view to illustrate the dimensions from every view.

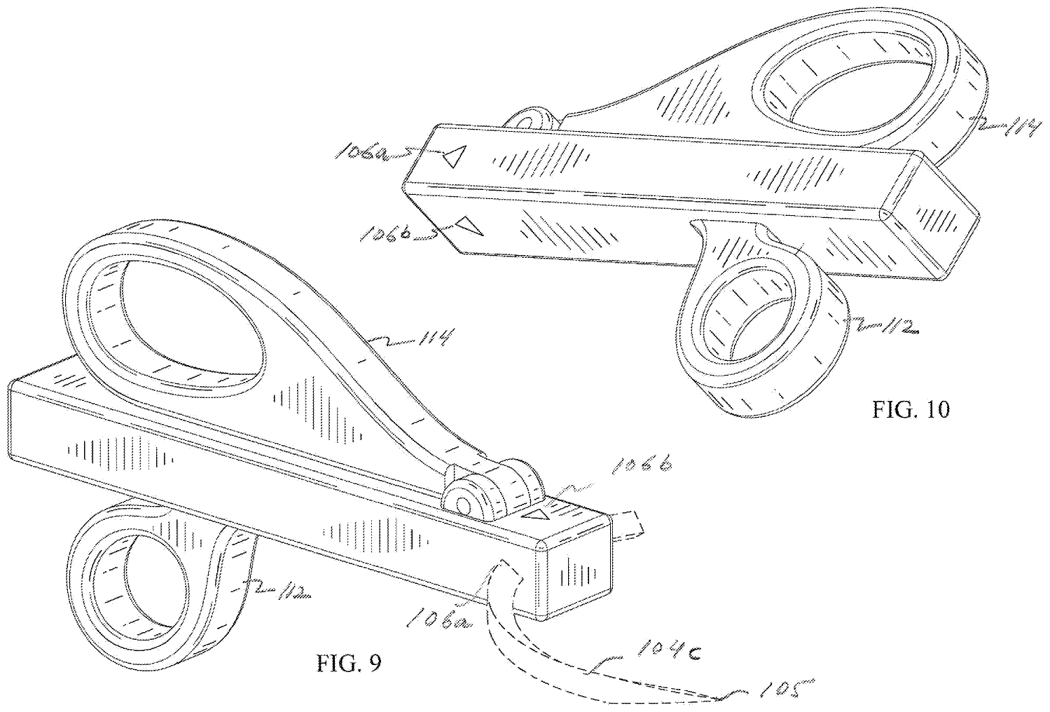

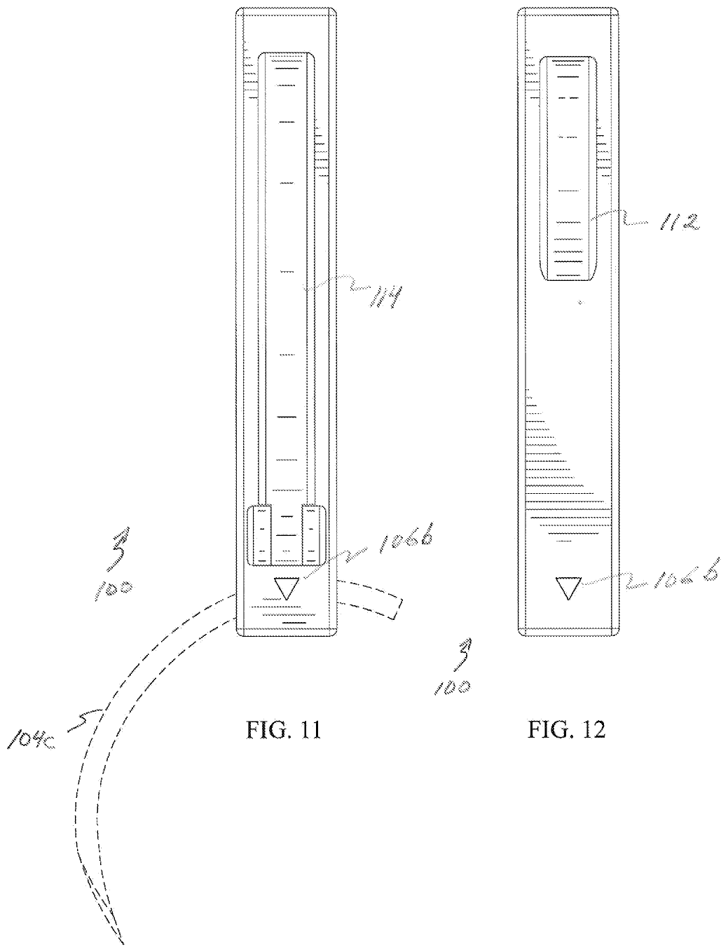

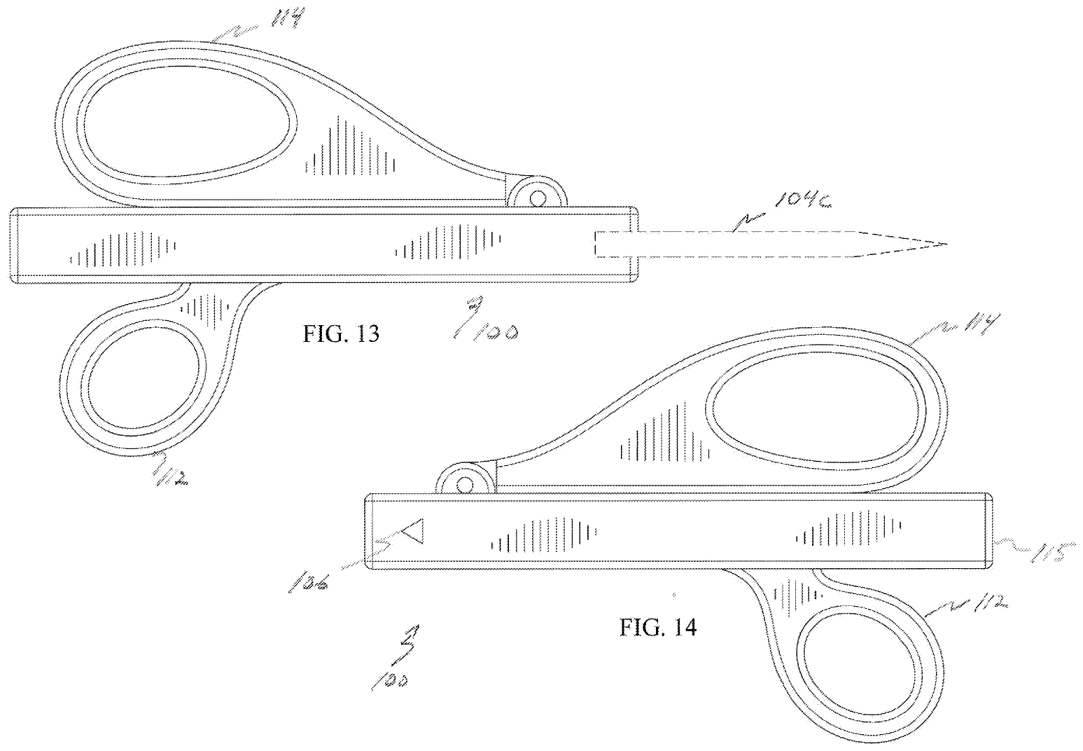

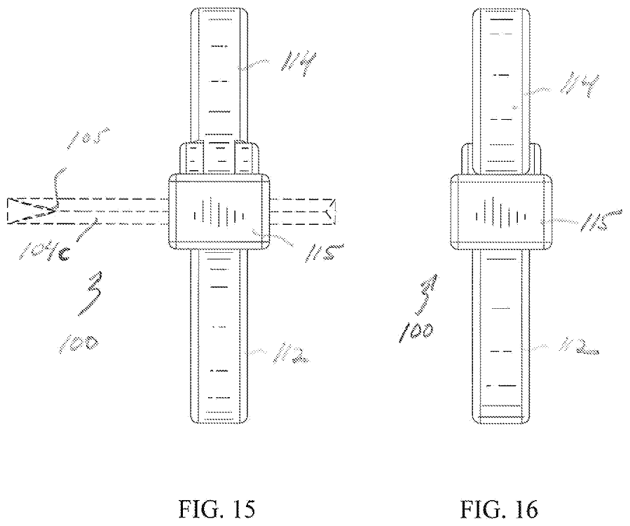

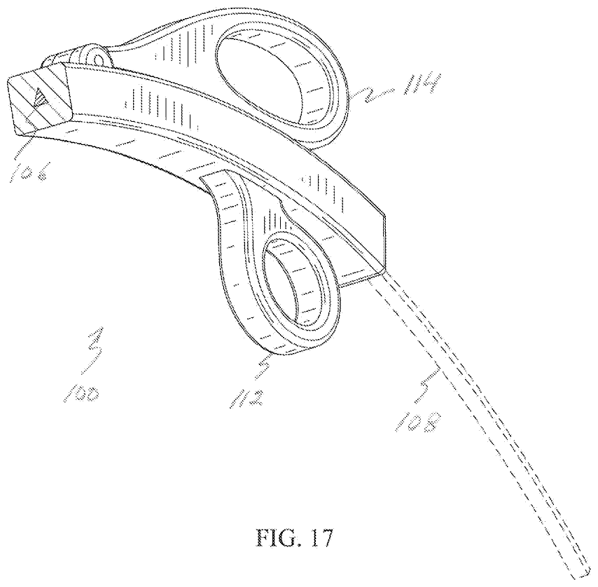

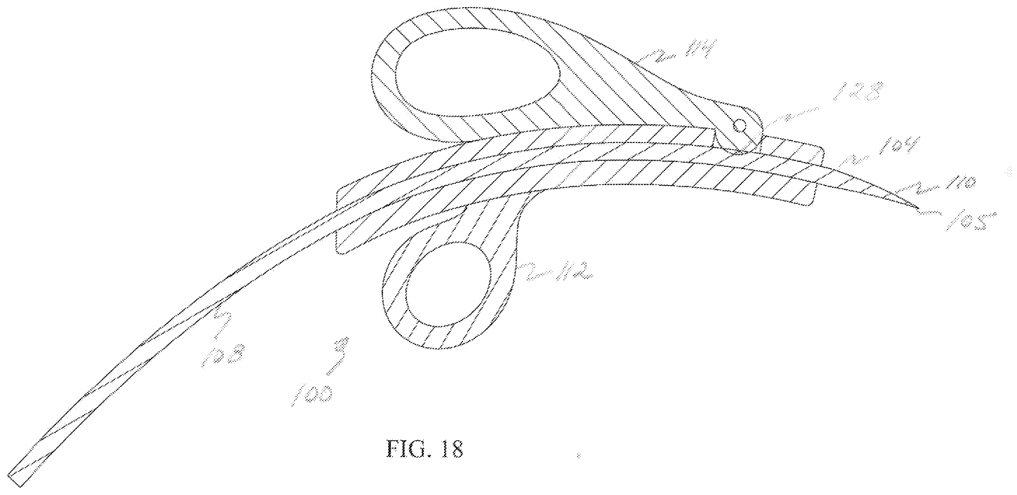

Referring now to FIGS. 9 and 10, another embodiment of the trocar device 102 is illustrated, where the trocar shaft 104c enters through an orifice 106a configured one the side. FIG. 10 is another view illustrating that in yet other embodiments, multiple orifices 106a and 106b on the side may be formed to receive different shapes of trocar shafts 104. FIGS. 11 and 12 illustrate planar views to illustrate the dimensions of the scissor members 112 and 114. The trocar shaft is 104c is shown through the orifice 106a (shown in FIG. 9). FIGS. 13 and 14 are yet additional side views illustrating the dimensions and orifice 106a to accommodate the trocar shaft 104c (in FIG. 13). FIGS. 15 and 16 are additional views that demonstrate the dimensions of the trocar device 102 and the way in which the scissor members 112 and 114 are attached. The trocar shaft 104c is oriented going through the orifice 106a showing the tip 105. FIG. 17 illustrates a partial cross section to show the solid material and the orifice 106 through which the shaft 104 extends. FIG. 18 is a cross sectional view illustrating the dimensions of each component. Each scissor member 112 and 114 is configured with a retention device 128 to grip the trocar shaft 104 when the surgeon or robot arm brings the scissor members towards each other to cause the retention device 128 to grip the trocar shaft 104.

In all the illustrated embodiments, the external diameter of the trocar shaft 104 (needle element) is substantially constant along the length of the trocar shaft 104 and the diameter of the channel 106 is substantially constant along the length of the sheath element. The surgical pin at the pivot axis 127 may be made of stainless steel and the sheath element may be made from medical grade plastic selected from the group consisting of urethane, polyethylene and polypropylene.

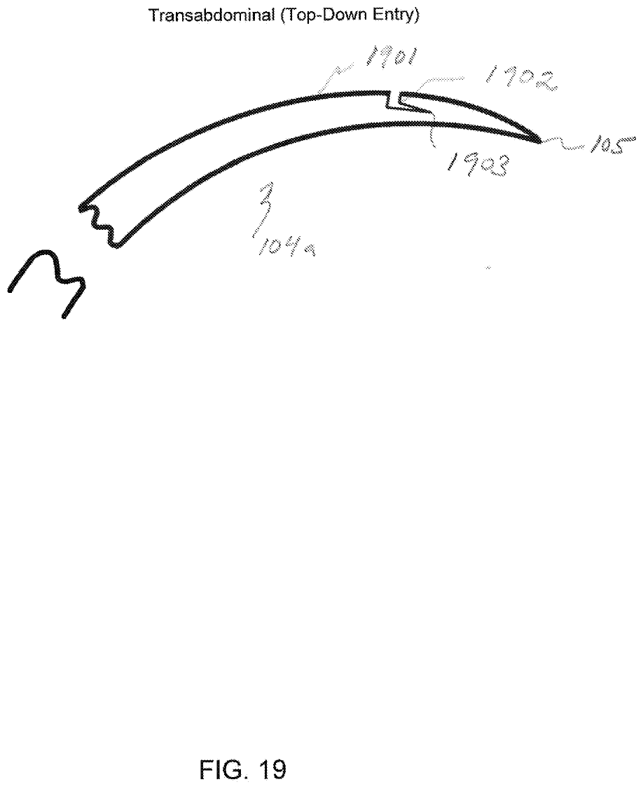

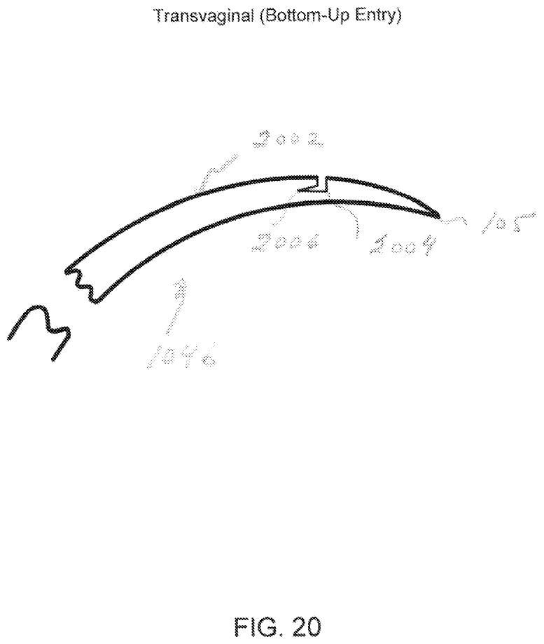

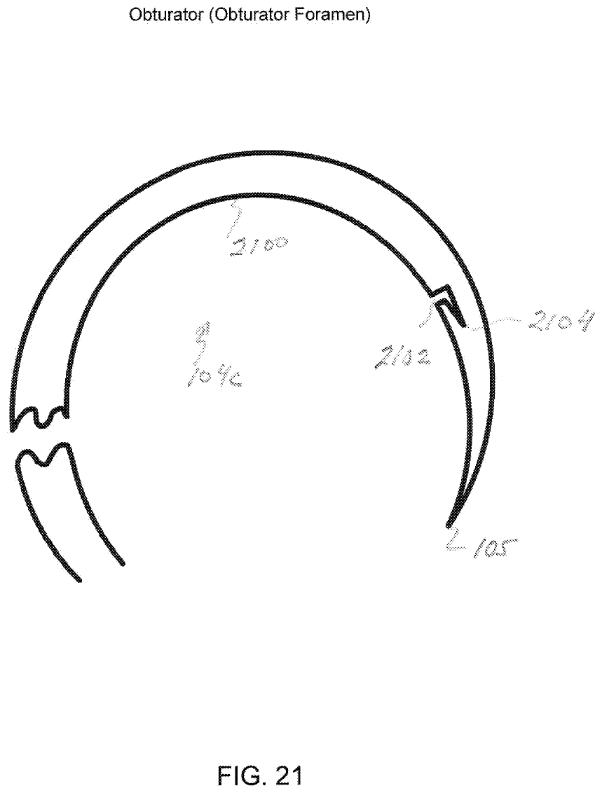

FIGS. 19 through 21 illustrate the different types of trocar shafts designated generally by reference numeral 104 for the different types of surgical procedures. FIG. 19 illustrates a particular curved trocar shaft designated by reference numeral 104a configured for transabdominal or top-down entry. The outer surface 1901 has a recess 1902 formed and configured with a tapered end extending toward the tip 105. FIG. 20 illustrates another type of curved trocar shaft 104b formed and configured for a transvaginal or bottom-up entry. The outer surface 2002 has a recess 2004 with a tapered end 2006 extending in the opposite direction from the tip 105. FIG. 21 illustrates yet another type of curved trocar shaft (obturator) designated by reference numeral 104c for the obturator foramen, where the trocar shaft 104c is configured to penetrate, hug the bone, and come out into the vagina. The inner surface 2100 has a recess 2102 with a tapered end 2104 extending toward the tip 105.

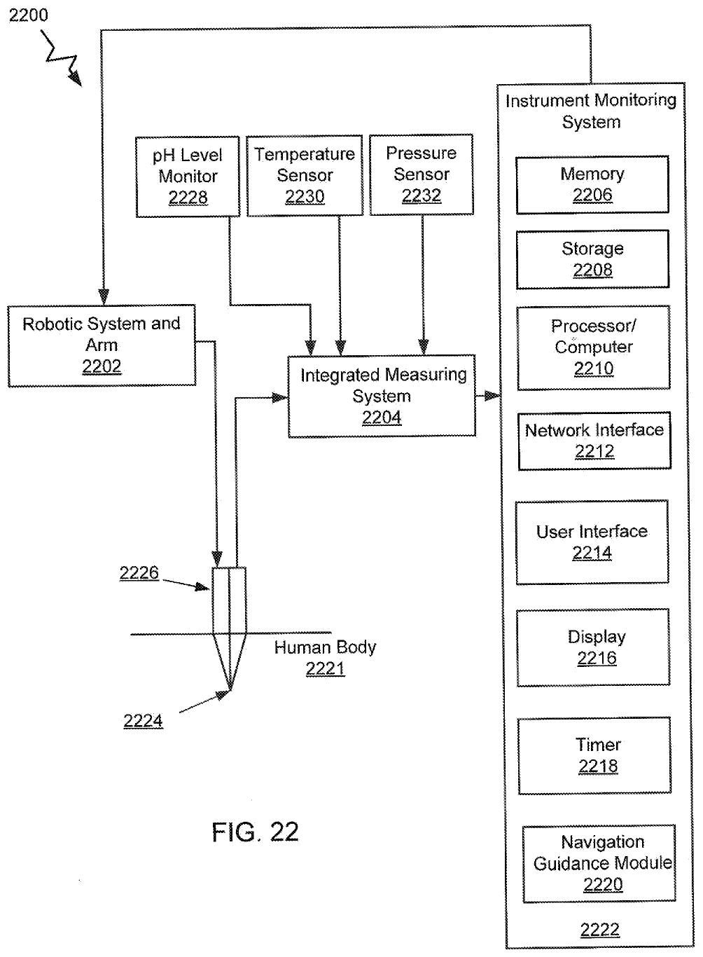

FIG. 22 illustrates a high-level system diagram designated by reference numeral 2200 of the improved trocar device 100 in accordance with any of the embodiments illustrated in the preceding figures (FIGS. 1-21), collectively illustrated by a graphical representation of the improved trocar device, which is designated by reference numeral 2226, and coupled to an instrument (e.g., trocar) monitoring system 2222. The graphical representation of the trocar device 2226 is illustrated as penetrating into a human body 2221, for example, via a vaginal cavity of a patient or human body 2222 or an abdominal surface of the patient or human body for surgical procedures. The trocar device or instrument 2226 may be introduced through the cavity and is guided or navigated by the surgeon or by a robot arm. Alternatively, a surgeon may work in conjunction with a robotic system. As illustrated, the other end of the trocar device 2226 (partially depicted) is typically held by a surgeon or the robotic arm coupled to a robotic system (together illustrated as block 2202). The robotic system comprises a controller (not separately shown) to guide the robot arm. It should be recognized by those skilled in the art that surgical robots give doctors more precision, flexibility and control. In some embodiments, the robot arm may consist of a camera. In addition, the robot arm may connect to a control console from which the surgeon can control the surgical procedure via the controller.

The tip 2224 (105 in FIGS. 1-21) of the trocar device 2226 that is inside the vaginal cavity is not visible from the outside, therefore, the surgeon may have difficulty determining the penetration path or the depth of the trocar device 2226 as it is navigated within a patient's body 2221. The penetration depth may be defined as the spacing of the distal end of the trocar device 2226 from the surface of the body (abdominal wall) of the patient. In some embodiments, the trocar device 2226 has the tip 2224 configured to hold a sensor or monitoring device. In some embodiments, a pressure sensor 2232 of suitable size is embedded at the tip 2224. The pressure sensor 2232 may be of miniature type that can be mass produced at low cost through microelectromechanical systems fabrication. In some embodiments, the pressure sensor 2232 is configured with a thin membrane that is flexible and can deform. The membrane covers a reference cavity. The reference cavity is preferably sealed at a low vacuum pressure. When there is a higher pressure outside than inside the reference cavity, the membrane stretches or deforms into the reference cavity. The membrane is attached to a rigid frame, so that all the deformation occurs in the membrane and not the frame. The deformation of the membrane causes two mechanical changes that are used for transduction to an electrical measurement. First, the membrane moves relative to the bottom of the cavity, which may be measured with a capacitance circuit. Second, the membrane materials experiences stress and strain, which may be measured with a resistive circuit. The displacement, stress, or strain in the membrane may be calculated by mathematical equations in ways that are known to those skilled in the art.

In accordance with one electrical measurement approach, to measure the displacement of the membrane, the sensor may be integrated into a capacitive measurement circuit. Electrodes are built into the top membrane and the bottom surface of the reference cavity. These electrodes act as a parallel plate capacitor. The capacitance between the plates is equal to C=eₒeᵣ*A/d where e₀ is the electrical permittivity of free space, eᵣ is the relative permittivity, A is the area of the plate, and d is the distance between the plates. As the membrane deforms, the distance, d, decreases, which increases the capacitance. A variety of electrical circuits may be used to convert the change in capacitance to a change in voltage which can be measured and converted into a digital signal.

In accordance with another electrical measurement approach, the strain in the membrane is measured. Any conductive material, like metal, changes resistance when a stress or strain is applied to it, therefore, such devices are referred to as strain gauges. Semiconductor materials, like doped silicon, experience a large change in resistance due to strain because of a material property called, piezoresistance. A large change in resistance is advantageous for the sensor design in the present system, therefore, a pressure sensor that uses a piezoresistive strain gauge to convert the mechanical forces into electrical changes is useful. In one configuration, four piezoresistors are configured around the membrane and connected in a circuit referred to as a Wheatstone bridge. The Wheatstone bridge increases the measurement signal and decreases sensor error from things like temperature change and other mechanical stresses. The Wheatstone bridge signal is amplified and then converted to a digital signal.

Pressure measurements are automatically transmitted at predetermined intervals to an integrated measuring system 2204. In some embodiments, for early detection of bladder perforation, a pH level monitor 2228 may be embedded or placed in at the tip 2224. A clue that a perforation of the bladder has taken place is by discovering via the pH level monitor 2228 that there is cystoscopic fluid extravasation around the trocar device 2226 or the sling material at the level of the abdominal skin. In some embodiments, a temperature sensor 2230 may be positioned at or near the tip 2224 to provide temperature readings. Other types of biometric sensors may be used to provide measurements beneficial to a surgeon or the robotic system 2202. All the measurements (by electronic signals) are transmitted to the integrated measuring system, from where data is dynamically transmitted to an instrument monitoring system 2222. In the event only a single sensor type is used, the integrated measuring system 2204 represents a measuring system for the single data desired.

The instrument monitoring system 2222, further comprises a memory 2206, a storage 2208, one or more processors 2210, a network interface 2212, a user interface 2214, a display 2216, a timer 2218, and a navigation guidance module 2222. The instrument monitoring system 2222 is configured to sense the path of the trocar device as it is navigated by the surgeon or robot arm.

The instrument monitoring system 2222 may take the form of an entirely hardware embodiment, an entirely software embodiment or an embodiment containing both hardware and software elements. In some embodiments, the instrument monitoring system 2222 is implemented in software, which includes but is not limited to firmware, resident software, microcode, etc. Furthermore, the instrument monitoring system 2222 may take the form of a computer program product accessible from a computer-usable or computer-readable medium providing program code for use by or in connection with a computer or any instruction execution system. For the purposes of this description, a computer-usable or computer readable medium may be any apparatus that can contain, store, communicate, propagate, or transport the program for use by or in connection with the instruction execution system, apparatus, or device.

A data processing system suitable for storing and/or executing program code is illustrated as at least one processor or computer 2210 coupled directly or indirectly to 2206 memory elements through a system bus (not shown). The memory elements 2206 may include local memory utilized during actual execution of the program code, bulk storage, cloud-based memory systems, and cache memories which provide temporary storage of at least some program code in order to reduce the number of times code must be retrieved from bulk storage during execution.

The instrument monitoring system 2222 may be implemented as a computer program executed by the processor 2210 (e.g., one or more processors arranged in a distributed architecture). Such a computer program may be stored in a computer readable storage medium (e.g., storage 2208), such as, but not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory, cloud-based systems, or any type of media suitable for storing electronic instructions, each coupled to a computer system bus.

The user interface 2214 comprises of input/output or I/O devices (including but not limited to keyboards, displays, pointing devices, etc.), which may be coupled to the system either directly or through intervening I/O controllers.

The network interface 2212 may comprise network adapters coupled to the system to enable the data processing system to become coupled to other data processing systems or remote printers or storage devices through intervening private or public networks (not shown). Modems, wireless modems, cable modem and Ethernet cards are just a few of the currently available types of network adapters. The network interface 2212 or the user interface 2214 are configured to connect to a display 2216 to display measurements in real time to, which may be displayed to the surgeon or any other system (remote or on location). The instrument monitoring system 2222 includes a timer 2218, which may control the predetermined time intervals for transmitting information to remote locations. In some instances, the timer 2218 is configured to interact with the memory 2206 or storage 2208 to record data transmitted by the integrated measuring system 2204. Measurements of pressure (or force) are dynamically transmitted as the trocar device 2226 probes into the patient's body (human body 2222). In addition, temperature measurement and measurements of pH levels may also be dynamically transmitted. The integrated measuring system 2204 comprises software modules for receiving measurements (of pH levels, temperature or pressure) in real time and processing them as described in greater detail below.

The navigation guidance module 2220 is a software module with executable code programmed to provide navigation or guidance data to the surgeon or robotic system controlling the surgical procedure. The navigation guidance module 2220 receives the measurements from the integrated measuring system 2204 (continuously or in predetermined time intervals) and in some instances transmits the measurements compiled in suitable form to the robotic system and arm 2202. In other instances, the measurements may be provided to the surgeon. The output may be in the form of continuous data feeds or formulated as alerts or other ways known to those skilled in the art.

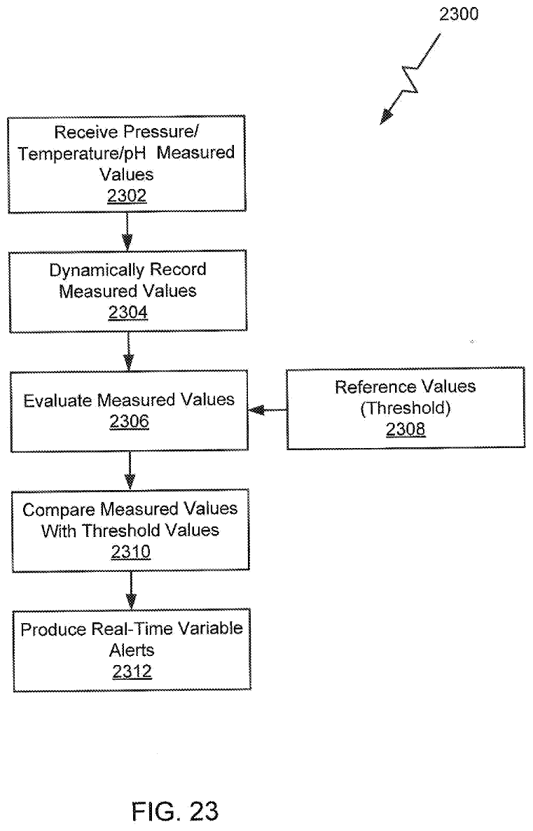

Referring now to FIG. 23, the instrument measuring system 2204 coupled to the instrument monitoring system 2222 performs a process designated generally by reference numeral 2300. The process 2300 begins at block 2302 representing one or more operations performed by executable code stored in either of the memory 2206 or storage 2208 to receive measured values from the pressure sensor 2232, the temperature sensor 2230, and/or the pH level monitor 2228. The process 2300 proceeds to the next block 2304 representing one or more operations performed by executable code for dynamically recording the measured values. In some embodiments, the measured values may be dynamically provided every minute or second or at other predetermined time intervals desired for the surgical procedure. The process 2300 proceeds to the next block 2306 of operations performed by executable code for evaluating the measured values. In some embodiments, the measured values may be evaluated by decisioning criteria processed by the processor 2210. In some embodiments, the block 2308 representing one or operations for providing reference values, for example, threshold values indicating critical levels, performs functions by executable code for providing the reference values. In some embodiments, the reference values may be obtained from active libraries of data, systems, websites, databases, or the like, by using filters to sort and select the data used to generate the reference values. The decisioning criteria in block 2306 transmits the measured values and select threshold values to the next block 2310 including one or more operations performed by executable code. The process 2300 continues to the next block 2312, including one or more operations for producing real-time variable alerts. In some embodiments, alerts may be displayed to the surgeon or the robotic system and arm 2202 to caution of critical situations. In other embodiments, audio alerts or other signals may be transmitted to the surgeon or the robotic system and arm 2202. For example, an indication may be formed by an output on a monitor or another display unit (e.g., touchpad, etc.). An alarm sound or a visual alarm signal (e.g., flashing, color indication) or a haptic signal (e.g., vibration of a lever) may also be output.

Finally, the algorithms and displays presented herein are not inherently related to any particular computer or other apparatus. Various general-purpose systems may be adapted for use with special programs in accordance with the teachings herein, or it may prove convenient to construct more specialized apparatuses to perform the required method steps. The required structure for a variety of these systems will appear from the description below. In addition, the present invention is not described with reference to any particular programming language. It will be appreciated that a variety of programming languages may be used to implement the teachings of the invention as described herein.

In some embodiments, the instrument monitoring method described in FIGS. 22 and 23 may be carried out continuously or at regular intervals after the beginning of the surgical procedure. In some scenarios, an expedient benefit is provided by recording one or more corresponding reference value(s) at the beginning of the surgical method to be able to reliably deduce changes. In block 2304, at least one measured value is recorded. The recorded value may allow the determination of a change in a force effect in the body as the trocar device 2226 (in FIG. 22) probes within the body of the patient. Multiple measured values may also be recorded. Examples of such measured values may include the penetration depth of the trocar device or instrument, the pressure in the body cavity of the patient, or the torque value that may be determined on at least one shaft of the robotic arm when used. Measured values may also be used directly or indirectly for determining pivot points for the instrument/trocar to guide the surgeon.

In some embodiments, after determining the measured value, the automatic evaluation is performed in block 2306 with respect to the previously recorded reference value, in particular by the processor 2210 or a system controller. A change in measured value for example may easily be determined, although a change in the force effect within the body may also be determined. In block 2310, the change in measured value or the change in the force effect is then compared with a threshold value provided by block 2308. The threshold value may be fixed in advance by a user or be empirically chosen by the system. In block 2312, an indication, alert or signal is automatically output if the threshold value has been detected as having been exceeded.

Reference will be made below to the specific designs of the method in relation to the recorded measured values and further examples will be described in detail. Multiple examples may also be used simultaneously.

Use of the penetration depth as a measured value may be made by determining the penetration depth of the trocar device or instrument 2226 in the abdominal cavity. The robotic arm 2202 may determine where the pivot point of the system for the trocar device or the instrument is located within the body. The penetration depth may also be measured by the integrated measuring system 2204 based on visual markers that may be indicated (e.g., length scales if desired and observed by a camera positioned for viewing). Each of the sensors illustrated and located on the tip 2224 of the trocar device 2226 reads the measured values continuously or at predetermined intervals. The integrated measuring system 2204 may automatically determine the length of the instrument and the penetration depth on the basis of the markings as additional data points for the decisioning criteria if desired. If the integrated measuring system 2204 is no longer able to determine values because the markings on the instrument are covered, visual, acoustic or mechanical warnings may likewise be output to the operator without interrupting the work using the system. The marking of the instrument is designed such that the whole instrument need not be visible. The instrument may be safely moved by the robotic arm in all remaining degrees of freedom for the patient by the automatic determination, continual updating, and constant monitoring of the penetration depth and path of the trocar instrument 2226. The forces robotically applied by the surgeon to the trocar device or instrument 2226 are correctly implemented and may not lead to incorrect movement e.g., movements injuring to the patient. The automatic detection of movement of the trocar device or instrument 2226 may facilitate a change of instrument without significant calibration steps or value inputs to be performed before each use, increasing the flexibility of the system while simultaneously simplifying operation.

The pressure in the body cavity may be advantageously used as a measured value where continuously and dynamically monitoring the pressure in the patient's body after entering through the vaginal cavity constitutes a significant safety measure for being able to react to unforeseen deformations or developments, and therefore, providing guidance for effecting changes. The pressure may change significantly if the trocar device 2226 slips or other movements are performed. Monitoring of the applied and effective forces is made possible hereby in the case of all movements running orthogonally to the pivot point. Monitoring the forces is an advantageous safety measure for avoiding movements of the instruments that are dangerous to the patient. In the event a robotic arm is used (see 2202), existing robotic arm torque sensors may be configured and used for monitoring and providing additional data. An advantage for each patient is provided if an initial pivot point (patient entry point) is defined at the beginning of the method. Events that may potentially change the pivot point translationally are changes in air pressure in the patient, deformations of internal organs, or deformations due to external effects.

If the automatic method finds that changes have occurred, timely countermeasures may be taken to prevent injury to the advantage of the patient. Monitoring the forces applied during movement of the robotic arm 2202, specifically in the pivot point of the instrument in the abdominal wall, is advantageous since injuries to the patient may otherwise occur. Forces may either be produced by the robotic arm 2202 or are exerted by the surgeon on the robotic arm 2202 as a movement control. If the robotic arm 2202 discovers a situation, corresponding signaling may occur and advantages are provided by limiting the force.

Reference in the specification to “one implementation or embodiment” or “an implementation or embodiment” simply means that a particular feature, structure, or characteristic described in connection with the implementation or embodiment is included in at least one implementation or embodiment of the technology described. The appearances of the phrase “in one implementation or embodiment” in various places in the specification are not necessarily all referring to the same implementation or embodiment.

The above description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof.

What is claimed is:

1. A surgical instrument, comprising:
a flexible introducer component, the flexible introducer component including two scissor members configured to allow a surgeon or a robot arm to hold the scissor members, the flexible introducer component including a channel opening extending through its length, from a distal end to a proximal end; and
a shaft component configured to be insertable within the channel opening, the shaft component movable within the channel opening of the flexible introducer component until the scissor members are compressed to cause a locking member of at least one of the scissor members to engage and grip the shaft component and arrest movement thereof.

2. The surgical instrument according to claim 1, further comprising:
at least one of pressure sensor, temperature sensor, and pH level monitor positioned at the tip of the shaft component, the pressure sensor, the temperature sensor, and the pH level monitor configured to provide measurements taken at predetermined intervals to a measurement system.

3. The surgical instrument according to claim 2, further comprising:
an instrument monitoring system coupled to the measurement system and configured to receive measurements from the measurement system, the instrument monitoring system further comprising a navigation guidance module configured to provide data to guide movement of the surgical instrument within a human body.

4. The surgical instrument according to claim 3, wherein the navigation guidance module provides real time navigation data to a surgeon or a robot arm.

5. The surgical instrument according to claim 4, wherein the robot arm is coupled to a robotic system with a controller for guiding the robot arm.

6. The surgical instrument according to claim 5, wherein the controller receives an output from the instrument monitoring system.

7. The surgical instrument according to claim 1, wherein the instrument monitoring system further comprises a navigation guidance module configured to provide data to guide movement of the surgical instrument within a human body.

8. The surgical instrument according to claim 1, wherein the scissor members include one large member and one small member and the locking mechanism is affixed to the large member.

9. The surgical instrument according to claim 1, wherein the locking mechanism affixed to the large scissor member has a protruding member configured to grip the shaft component as the introducer component is moved up and down on the shaft component by a surgeon's hand or a robot's arm.

10. The surgical instrument according to claim 1, wherein said flexible introducer component is made from a bio-compatible plastic material taken from a group including acetal, polyamide, polyethylene, polyvinylchloride; said plastic material being sterilizable at high temperature.

11. A method for constructing a surgical instrument for navigation into a human body,
comprising:
forming a flexible introducer component, the flexible introducer component including two scissor members configured to allow a surgeon or a robot arm to hold the scissor members, the flexible introducer component including a channel opening extending through its length, from a distal end to a proximal end; and
inserting a shaft component within the channel opening, the shaft component movable within the channel opening of the flexible introducer component until the scissor members are compressed to cause a locking member of at least one of the scissor members to engage and grip the shaft component and arrest movement thereof.

12. A method according to claim 11, further comprising:
positioning at least one of a pressure sensor, a temperature sensor, and a pH level monitor at the tip of the shaft component, with the pressure sensor, the temperature sensor, and the pH level monitor providing a measurement taken at predetermined intervals;
coupling a measurement system to the at least one of the pressure sensor, the temperature sensor and the pH level monitor to track the measurements.

13. The method according to claim 12, further comprising:
coupling an instrument monitoring system to the measurement system to receive measurements from the measurement system, the instrument monitoring system further comprising a navigation guidance module to provide data to guide movement of the surgical instrument within a human body.

14. The method according to claim 13, wherein the navigation guidance module provides real time navigation data to a surgeon or a robot arm.

15. The method according to claim 14, wherein the robot arm is coupled to a robotic system with a controller for guiding the robot arm.

16. The method according to claim 15, wherein the controller receives an output from the instrument monitoring system.

17. The method according to claim 11, wherein the instrument monitoring system further comprises a navigation guidance module configured to provide data to guide movement of the surgical instrument within a human body.

18. The method according to claim 11, wherein the scissor members include one large member and one small member and the locking mechanism is affixed to the large member.

19. The method according to claim 11, wherein the locking mechanism affixed to the large scissor member has a protruding member to grip the shaft component as the introducer component is moved up and down on the shaft component by a surgeon's hand or a robot's arm.

20. The method according to claim 11, wherein said flexible introducer component is made from a bio-compatible plastic material taken from a group including acetal, polyamide, polyethylene, polyvinylchloride; said plastic material being sterilizable at high temperature.
